(19) United States
(12) Patent Application Publication
(10) Pub. No.: US 2006/0110351 A1
(43) Pub. Date: May 25, 2006

(54) Use of silanes on cosmetic agents and methods for treating hair

(76) Inventors: Sabine Koehler, Mandelbachtal (DE); Juergen Allwohn, Burgschwalbach (DE); Ludwig Meitzler, Weinheim (DE); Ralph Nonninger, Saarbruecken (DE); Uwe Carl Schmidt, Reinheim (DE)

Correspondence Address: Striker Striker & Stenby, 103 East Neck Road, Huntington, NY, 11743, US

(21) Appl. No.: 10/521,791

(22) PCT Filed: Jul. 15, 2003
(86) PCT No.: PCT/EP03/07626

§ 371 Date: Aug. 11, 2005

(30) Foreign Application Priority Data
Jul. 25, 2002 (DE) 102 33 963.5

(51) Int. Cl. A61K 8/89

(52) U.S. Cl. 424/70.12; 8/406

(57) ABSTRACT

The cosmetic agent contains from 0.01 to 10 percent by weight of at least one silane of formula RₘSiX₄₋ₘ, wherein m=0 to 4, R is a nonhydrolyzable organic group and X is a hydrolysable group, or a salt thereof. The method for improving the condition of hair, particularly in terms of protection, hardening, strengthening, sealing, restructuring, repairing, stabilizing, enhancing the luster, facilitating the combability and shortening the hair-drying time, includes bringing the aforesaid cosmetic agent into contact with the hair for a period of 10 seconds to 60 minutes at a temperature between 20° C. and 60° C.

The present invention has for an object the use of an agent containing at least one silane for improving the condition of hair, particularly for sealing, hardening, luster improvement, strengthening, protecting as well as improving the structure (restructuring) of human hair, preferably damaged human hair.

That human hair can be damaged by environmental factors (for example energy-rich radiation) or as a result of physiological status (for example age or health of the individual involved) or mechanical and chemical effects is known. The damage results in deleterious mechanical, chemical and physical properties of the affected hair. Such damage to the hair structure manifests itself, for example, as a loss of hair, luster, thickness, breaking strength, tear strength, bundle tensile strength and color retention capacity.

In the case of human hair, such effects manifest themselves particularly as loss of luster, reduced tear strength and poor combability. These effects are a result of aging processes brought about primarily physiologically or induced by physical (weathering), mechanical (combing, brushing, rubbing) and chemical factors. In long hair, these effects become apparent particularly at the hair tips (splits). The chemical factors include, in particular, bleaching, oxidative dyeing and permanent waving of the hair for which aggressive oxidants or reducing agents are used, preferably in a strongly alkaline environment in which they show their full effect. Other chemical agents, for example water enriched in chlorine or salts, however also exert a harmful effect on keratin-containing material.

Commercial rinses and conditioners contain as active substances mainly cationic surfactants or polymers, waxes and/or oils. The more damaged the hair, the greater is the amount of anionic groups present on the surface. Cationic compounds are attracted by said surface electrostatically, because said surface bears an electric charge of opposite sign, whereas oils and waxes interact with the hydrophobic groups of the keratin. Hence, an improvement in the internal hair structure or an effective sealing of the hair cannot be achieved with these hair-care products.

Previously dyed hair often shows color loss owing to washout, perspiration, exposure to light and permanent wave treatments. Effective sealing of the hair after the dyeing process therefore would be desirable. Hence, a further object of the invention was to provide a hair treatment agent that, particularly after hair dyeing, seals the hair in a manner such that the dye is no longer readily washed out.

The objective underlying the present invention was to provide an agent, particularly a cosmetic hair treatment agent, to be used for the improvement of the condition of hair and for providing effective protection or sealing so as to eliminate the afore-indicated drawbacks.

According to the invention, these objectives are reached by the use of at least one silane in a cosmetic agent for improving the condition of hair.

The improvement in the condition of hair consists preferably of hardening, strengthening, sealing (particularly after hair dyeing or hair tinting) restructuring, repairing, stabilizing, enhancing the luster, volume and combability, protecting from environmental influences, protecting from heat during hair drying (hot air produced by a hair drier, hair smoothing with a hot iron), preventing and reducing hair splitting, shortening the time for hair drying and increasing the extensibility and elasticity of hair as well as the permanence of permanent waves.

Surprisingly, we have found that by use of certain silanes described hereinbelow the hair structure overall, but particularly at the surface of the hair, is changed so that, as a result, protection, hardening, sealing and strengthening as well as an increase in breaking strength, tear strength or bundle tensile strength is achieved, particularly on weakened and damaged hair.

In addition to the hair-care action described hereinbelow and resulting from an effect on the hair surface (cuticula), a repairing action is also achieved. Said action is due to changes within the hair (cortex). We have measured tensile forces that cause oxidatively predamaged (bleached) hair to tear. Surprisingly, we have found that hair which after oxidative damage had been treated with an agent containing one of the herein-described silanes showed a significant increase in the forces required to cause tearing.

In connection therewith, not only can damaged hair be restructured (repaired), but a protective effect is possible as well, an effect that counteracts damage to the hair before or during exposure to the said noxae thus preventing or reducing it.

Besides these disadvantageous changes induced by exogenous noxae, the agent of the invention can bring about advantageous effects also as a result of conditions or changes in the hair structure induced by physiological processes, for example in brittle hair resulting from age or in fine hair acquired congenitally or as a result of age (baby hair, old-age hair).

The present invention therefore has for an object the use according to claim 1. The preferably used silanes are indicated in claims 2 to 5. More preferably used silanes are those indicated in claims 6 to 10, the silanes according to claims 9 and 10 being especially preferred.

The following silanes of formula (I) are suitable for use according to the invention:
3-(trimethoxysilyl)propyldimethyloctadecylammonium chloride,
3-(trimethoxysilyl)propylmethyldi(decyl)ammonium cloride,
3-chloropropyltrimethylsilane, octadecyltrimethoxysilane,
methyldifluorosilane, dimethyldifluorosilane, methyltrifluorosilane,
dimethyldifluorosilane, dimethylfluorosilane, trimethylfluorosilane,
ethyldimethylfluorosilane, ethyltrifluorosilane,
triethylfluorosilane, triphenylfluorosilane,
diphenyldifluorosi lane, pentafluorophenyldimethylchlorosi lane,
phenyltrifluorosi lane, triphenylfluorosi lane,
trifluoromethyltrimethylsilane, hexyltrifluorosilane,
bis(pentafluorophenyl)dimethylsilane, (trifluoromethyl)trimethylsilane, trifluoromethyltrimethylsilane,
vinyidimethylfluorosilane, tetrafluorosilane, trifluoromethyltriethylsilane,
trifluoromethyltrimethylsilane [sic], chloromethyldimethylfluorosilane,
3-(heptafluoroisopropoxy)propyltrichlorosilane,
3-(heptafluoroisopropoxy)propyltriethoxysilane, hexafluorodisilane, triethoxyfluorosilane,
1H,1H,2H,2H-perfluorodecyldimethylchlorosilane,
1H,1H,2H,2H-perfluorodecyltrichlorosilane,
1H,1H,2H,2H-perfluorodecyltriethoxysilane,
1H,1H,2H,2H-perfluorooctyltriethoxysilane,
1H,1H,2H,2H-perfluorooctyldimethylchlorosilane,
1H,1H,2H,2H-perfluorooctylmethyldichlorosilane,
1H,1H,2H,2H-perfluorooctyltrichlorosilane,
1H,1H,2H,2H-perfluorooctylmethyldimethoxysilane,
1H,1H,2H,2H-perfluorotetradecyltriethoxysilane,
(3,3,3-rifluoropropyl)dimethylchlorosilane,
(3,3,3-trifluoropropyl)dichloromethylsilane,
(3,3,3-trifluoropropyl)methyldimethoxsilane,
(3,3,3-trifluoropropyl)trichlorosilane,
(3,3,3-trifluoropropyl)trimethoxysilane,
bis(tridecafluoro-1,1,2,2-tetrahydrooctyl)dimethylsiloxymethylchlorosilane,
(3-heptafluoroisopropoxy)propyltrichlorosilane,
(heptadecafluoro-1,1,2,2-tetrahydrodecyl)dimethylchlorosilane,
(heptadecafluoro-1,1,2,2-tetrahydrodecyl)methyldichlorosilane,
(heptadecafluoro-1,1,2,2-tetrahydrodecyl)tiethoxysilane,
pentafluorophenyldimethylchlorosilane, pentafluorophenylpropyltrichlorosilane,
pentafluorophenylpropyldimethylchlorosilane, pentafluorophenyltrimethylsilane,
pentafluorophenylpropyltrimethoxysilane, pentafluorophenyltriethoxysilane,
pentafluorophenylpropylmethyldichlorosilane,
(3,3,3-trifluoropropyl)dimethylchlorosilane [sic], (3,3,3-trifluoropropyl)trichlorosilane [sic],
(3,3,3-trifluoropropyl)methyldichlorosilane, (3,3,3-trifluoropropyl)trimethoxysilane [sic],
trifluoropropyltris(dimethylsiloxy)silane, pentafluorophenylethoxydimethylsilane,
vinyl-(trifluoromethyl)dimethylsilane, vinyl-(3,3,3-trifluoropropyl)dimethylsilane.
(CH₃O)₃Si(CH₂)₃N⁺(CH₃)₂C₁₈H₃₇Cl⁻,
(CH₃O)₃Si(CH₂)₃N⁺(CH₃)₂C₁₈H₃₇Br⁻, (CH₃O)₃Si(CH₂)₃N⁺(CH₃)₂C₁₀H₂₁Cl⁻,
(CH₃O)₃Si(CH₂)₃N⁺(CH₃)₂C₁₀H₂₁Br⁻, (CH₃O)₃Si(CH₂)₃N⁺(CH₃)₃Cl⁻;
(CH₃O)₃Si(CH₂)₃N⁺(CH₃)₂C₈H₁₇Cl⁻, (CH₃O)₃Si(CH₂)₃N⁺(CH₃)₂C₁₀H₂₁Cl⁻,
(CH₃O)₃Si(CH₂)₃N⁺(CH₃)₂C₁₂H₂₅Cl⁻; (CH₃O)₃Si(CH₂)N⁺(CH₃)₂C₁₄H₂₉Cl⁻,
(CH₃O)₃Si(CH₂)₃N⁺(CH₃)₂C₁₆H₃₃Cl⁻, (CH₃O)₃Si(CH₂)₃N⁺(CH₃)₂C₂₀H₄₁Cl⁻,
(CH₃O)₃Si(CH₂)₃N⁺(C₄H₉)₃Cl⁻; (CH₃O)₃Si(CH₂)₃N⁺(C₂H₅)₃Cl⁻,
(CH₃CH₂O)₃Si(CH₂)₃N⁺(CH₃)₂C₁₈H₂₇Cl⁻, (CH₃O)₃Si(CH₂)₃NHC(O)(CF₂)₈CF₃,
(CH₃O)₃Si(CH₂)₃NHC(O)(CF₂)₈CF₃, (CH₃O)₃Si(CH₂)₃NHC(O)(CF₂)₁₀CF₃,
(CH₃O)₃Si(CH₂)₃NHC(O)(CF₂)₁₂CF₃, (CH₃O)₃Si(CH₂)₃NHC(O)(CF₂)₁₄CF₃,
(CH₃O)₃Si(CH₂)₃NHC(O)(CF₂)₁₆CF₃, (CH₃O)₃Si(CH₂)₃NHSO₂(CF₂)₇CF₃,
(CH₃O)₃Si(CH₂)₃N⁺(CH₃)₂(CH₂)₃NHC(O)(CH₂)₆CH₃,
(CH₃O)₃Si(CH₂)₃N⁺(CH₃)₂(CH₂)₃NHC(O)(CH₂)₈CH₃,
(CH₃O)₃Si(CH₂)₃N⁺(CH₃)₂(CH₂)₃NHC(O)(CH₂)₁₀CH₃,
(CH₃O)₃Si(CH₂)₃N⁺(CH₃)₂(CH₂)₃NHC(O)(CH₂)₁₂CH₃,
(CH₃O)₃Si(CH₂)₃N⁺(CH₃)₂(CH₂)₃NHC(O)(CH₂)₁₄CH₃,
(CH₃O)₃Si(CH₂)₃N⁺(CH₃)₂(CH₂)₃NHC(O)(CH₂)₁₆CH₃,
(CH₃O)₃Si(CH₂)₃N⁺(CH₃)₂(CH₂)₃NHC(O)(CH₂)₆CH₃,
(CH₃O)₃Si(CH₂)₃N⁺(CH₃)₂(CH₂)₃NHC(O)(CH₂)₈CH₃,
(CH₃O)₃Si(CH₂)₃N⁺(CH₃)₂(CH₂)₃NHC(O)(CH₂)₁₀CH₃,
(CH₃O)₃Si(CH₂)₃N⁺(CH₃)₂(CH₂)₃NHC(O)(CH₂)₁₂CH₃,
(CH₃O)₃Si(CH₂)₃N⁺(CH₃)₂(CH₂)₃NHC(O)(CH₂)₁₄CH₃,
(CH₃O)₃Si(CH₂)₃N⁺(CH₃)₂(CH₂)₃NHC(O)(CH₂)₁₆CH₃,
(CH₃O)₃Si(CH₂)₃N⁺(CH₃)₂(CH₂)₃NHSO₂(O)(CH₂)₇CH₃,
(CH₃O)₃Si(CH₂)₃N⁺(CH₃)₂(CH₂)₃NHSO₂(O)(CH₂)₉CH₃,
(CH₃O)₃Si(CH₂)₃N⁺(CH₃)₂(CH₂)₃NHSO₂(O)(CH₂)₁₁CH₃,
(CH₃O)₃Si(CH₂)₃N⁺(CH₃)₂(CH₂)₃NHSO₂(O)(CH₂)₁₃CH₃,
(CH₃O)₃Si(CH₂)₃N⁺(CH₃)₂(CH₂)₃NHSO₂(O)(CH₂)₁₅CH₃,
(CH₃O)₃Si(CH₂)₃N⁺(CH₃)₂(CH₂)₃NHSO₂(O)(CH₂)₁₆CH₃,
aminoethylaminopropyltrmethoxsilane: NH₂(CH₂)₂NH(CH₂)₃Si(OCH₃)₃,
3-aminopropyltrimethoxyslane: NH₂(CH₂)₃Si(OCH₃)₃,
3-amiinopropyltriethoxysilane: NH₂(CH₂)₃Si(OCH₂CH₃)₃,
3-hloropropyltrimethoxysilane: Cl(CH₂)₃Si(OCH₃)₃,
3-chloropropyltriethoxysilane: Cl(CH₂)₃Si(OCH₂CH₃)₃,
3-chloropropyltrichlorosilane: Cl(CH₂)₃SiCl₃,
3-glycidoxypropyltrimethoxysilane: C₃H₂O₂(CH₂)₃Si(OCH₃)₃,
3-glycidoxypropyltriethoxysilane: C₃H₅O₂(CH₂)₃Si(OCH₂CH₃)₃,
3-methacryloxypropyltrimethoxysilane: C₄H₅O₂(CH₂)₃Si(OCH₃)₃,
3-methacryloxypropyltriethoxysilane: C₄H₅O₂(CH₂)₃Si(OCH₂CH₃)₃,
methyldichlorosilane: CH₃SiHCl₂ silane-modified melamines: Dow Corning Q1-6106,
sodium (trhydroxysilyl)propylmethylphosphonate: NaO(CH₃)P(O)(CH₂)₃Si(OH)₃,
trichlorosilane: SiHCl₃, n-2-vinylbenzylaminoethyl-3-aminopropyltrimethoxysilane.HCl:
Dow Corning Z-6032,
vinyltriacetoxysilane: H₂C═CHSi(OCOCH₃)₃,
vinyltrimethoxysilane: H₂C═CHSi(OCH₃)₃,
vinyltriethoxysilane: H₂C═CHSi(OCH₂CH₃)₃, vinyltrihlorosilane: H₂C═CHSiCl₃,
dimethylidichlorosilane: (CH₃)₂SiCl₂ dimethyldimethoxysilane: (CH₃)₂Si(OCH₃)₂,
diphenyldichlorosilane: (C₆H₅)₂SiCl₂, ethyltrichorosilane: (C₂H₅)SiCl₃,
ethyltrimethoxysilane: (C₂H₅)Si(OCH₃)₃,
ethyltriethoxysilane: (C₂H₅)Si(OCH₂CH₃)₃, isobutyltrimethoxysilane,
n-octyltriethoxysilane, methylphenyldichlorosilane: CH₃(C₆H₅)SiCl₂,
methyltrichlorosilane: CH₃SiCl₃, methyltrimethoxysilane: CH₃Si(OCH₃)₃,
phenyltrichlorosilane: C₆H₃SiCl₃, phenyltrimethoxysilane: C₆H₅Si(OCH₃)₃,
n-propyltrichlorosilane: C₃H₇SiCl₃, n-propyltrimethoxysilane: C₃H₇Si(OCH₃)₃,
silicon tetrachloride: SiCl₄, ClCH₂C₆H₄CH₂CH₂SiCl₃,
ClCH₂C₆H₄CH₂CH₂Si(OCH₃)₃, ClCH₂C₆H₄CH₂CH₂Si(OCH₂CH₃),
decyltrichlorosilane, dichloromethyl(4-methylphenethyl)silane,
diethoxymethylphenylsilane, [3-(diethylamino)propyl]trimethoxysilane,
3-(dimethoxymethylsilyl)-1-propanethiol, dimethoxymethylvinylsilane,
3-[tris(trimethylsilyloxy)silyl]propyl methacrylate, trichloro[4-chloromethyl)-phenyl]silane,
methylbis(trimethylsilyloxy)vinylsilane, methyltripropoxysilane and trichlorocyclopentylsilane.

Optimum performance in terms of improving the condition of the hair is achieved with a silane substituted with at least one fluoroalkyl group and having the formula
(R_{c}O)₃SiR_{d} (IX)
wherein R_{c} is a staright-chain or branched alkyl group with 1 to 5 carbon atoms and Rd denotes a straight-chain or branched fluorinated or perfluorinated alkyl group with 1 to 18 carbon atoms and preferably with 8 to 10 carbon atoms.

The use of a fluoro compound as hydrophobing component is known, for example, from the production of low-energy surfaces as easy-to-clean surfaces which as a result of their reduced energy attract only very small amounts of dirt and can be cleaned with a minor cleaning effort. We have now found that these compounds can be used in the field of hair cosmetics to improve the condition of hair. In this case, a reduction in hair surface energy results in reduced deposition of dirt and grease. The hair thus feels clean longer. Moreover, it is possible to bring about the hydrophobing of hair by use of fluoroalkylsilane compounds to impart water repellency. Thus, the hair swells less, dries faster, and during humid weathering can be prevented from frizzing. In addition, the fluorosilanes exert a smoothing action. The combability of the hair can thus be enhanced, and the formation of brittle, dry tips can be prevented. All these actions improve the condition of the hair.

Unless otherwise indicated, in the present patent application the terms given in the following in quotation marks have the following meaning:

A “hydrolyzable group” is a group that in water or in an acidic aqueous solution hydrolyzes within a short time. Examples of such groups are F, Cl, Br, OH, acetyl, acetoxy, acyl, acyloxy and alkoxy with 1 to 6 carbon atoms.

“Alkyl” is a straight-chain or branched saturated hydrocarbon group with 1 to 24 carbon atoms, particularly methyl (“Me”), ethyl (“Et”), n-propyl, isopropyl, n-butyl, isobutyl, t-butyl, octyl, decyl, tetradecyl, hexadecyl, eicosyl and tetracosyl.

“Hydroxyalkyl” is an alkyl group with one or more hydroxyl groups, for example —CH₂CH₂OH, —CH₂CH(OH)CH₃, —CH₂OH, —CH₂CH₂CH₂OH, —CH₂CH₂CH(OH)CH₃, —CH₂CH₂CH(OH)CH₂OH or —CH₂CH(OH)CH(OH)CH₃.

“Alkoxy” is an alkyl group attached to a terminal oxygen atom.

“Polyalkyl ethers” are alcohols connected to each other through ether bonds. They can have an average molecular weight of up to 10,000 g/mol.

“Alkyl glycol” is a compound with an alkyl grouping linked to a glycol group through an ether bond, for example ethylene glycol monobutyl ether.

“Alkyl polyglycol” denotes a compound with an alkyl grouping linked to a polyglycol through an ether bond. The alkyl polyglycols should have an average molecular weight of up to 10,000 g/mol.

“Aryl” and “aromatic” are interchangeable and denote a molecule or a group in which the atoms form one or more rings with conjugated double bonds, particularly benzene, naphthalene, phenanthrene and anthracene. Examples of such groups are phenyl, benzyl, naphthyl, benzylidene, xylyl, styrene [sic], styryl, phenethyl and phenylene.

“Substituted” means that at least one hydrogen atom is replaced with a halogen, particularly Br, Cl or F, or with a hydroxyl group, alkyl group or alkoxy group.

“Branched” means a branched alkyl or alkoxy group, for example isobutyl, t-butyl, isopropyl, —CH₂CH₂C(CH₃)(H)CH₂CH₃, —CH₂C(CH₂CH₃)(H)CH₂CH₃, —CH₂CH₂C(CH₃)CH₃ and —CH₂CH₂C(CH₃)₃.

“Acyl” denotes the group derived from an organic acid and having the formula R′CO wherein R′ is an alkyl group.

“Acyloxy” denotes the group derived from an organic acid and having the formula R′COX′ wherein R′ denotes an alkyl group and X′ rather than denoting a hydroxyl group stands for an oxygen atom linked to some other group.

The term “perhalogenated” refers to a hydrocarbon group wherein all hydrogen atoms are replaced with F, Cl or Br. “Halogenated”, on the other hand, means that at least one hydrogen atom is replaced with F, Cl or Br.

The term “perfluoro” refers to a hydrocarbon group in which all hydrogen atoms are replaced with fluorine. “Fluorinated”, on the other hand, means that at least one hydrogen atom is replaced with fluorine.

Any mixtures of the silanes or of the salts thereof can be used for the indicated purpose.

Additional embodiments of the present invention are described in the claims.

The use of the silane consists of bringing the hair in contact with a silane-containing agent, preferably allowing said agent to remain on the hair or, after an appropriate exposure time, rinsing it off or washing it out with an aqueous agent.

In the agent used, the silane is preferably contained in an amount from 0.001 to 30.0 weight percent, more preferably in an amount from 0.1 to 10.0 wt. % and particularly in an amount from 0.1 to 2 wt. %, always based on the total amount of the agent.

The agent used according to the invention can be in the form of any suitable formulation known for hair cosmetics.

In particular, the agent can be an aqueous or aqueous-alcoholic solution, a gel, cream, emulsion or foam, and it can be formulated in the form of a one-component or a multicomponent preparation. In the case of a one-component preparation, the agent contains the silane together with appropriate cosmetic auxiliary agents and carriers (for example thickeners, acids, fragrances, solvents, salts, welting agents and/or UV absorbers).

If the agent is in the form of a multicomponent preparation, it can consist of at least two different components that are kept spatially separated until they are used. A first component can contain either only the silane (active ingredient) of the present invention or in this first component the active ingredient can be mixed together with an auxiliary agent (for example a thickener), advantageously in dry, solid form (for example as a powder in compressed or uncompressed form, as granulate or tablet). A second or additional component contains only auxiliary agents and carriers.

It is also possible, however, that in a multicomponent preparation different components contain different active ingredients according to the present invention individually or in admixture, either by themselves or together with various auxiliary agents, and the other components contain only auxiliary agents and carriers.

The invention comprises the use of a composition characterized in that it is a one-component preparation or a multicomponent preparation. If the agent used is a multicomponent preparation, it comprises a first component containing the silane with or without auxiliary agents and additives and a second component containing the remaining constituents. The agent used can also be a multicomponent preparation with at least three different components of which at least one contains the silane, the other components containing the remaining constituents.

To prepare the ready-to-use agent, the spatially separated individual components of a multicomponent preparation must, of course, be mixed with one another just before they are used according to the invention.

The agent used according to the invention can additionally contain carriers and auxiliary agents, for example solvents such as water, lower aliphatic alcohols, for example ethanol, n-propanol and isopropanol, furthermore dissolution promoters, wetting agents or emulsifiers from the classes of anionic, cationic, amphoteric or nonionic surface-active substances, such as fatty alcohol sulfates, ethoxylated fatty alcohol sulfates, alkylsulfonates, alkylbenzenesulfonates, alkyltrimethylammonium salts, alkylbetaines, ethoxylated fatty alcohols, ethoxylated nonylphenols, fatty alkanolamides, ethoxylated fatty alcohols [sic], ethoxylated nonylphenols [sic], fatty alkanolamides [sic], ethoxylated fatty esters, furthermore thickeners such as higher fatty alcohols, starch or cellulose derivatives; salts, for example NaCl; buffering agents such as ammonium hydrogen carbonate; thiols, ketocarboxylic acids (oxocarboxylic acids), particularly α-ketocarboxylic acids, or the physiologically compatible salts thereof, UV absorbers, perfumes, dyes, conditioners, hair swelling agents, preservatives, vaselines, paraffin oil and fatty acids, as well as hair-care agents such as cationic resins, lanolin derivatives, cholesterol, pantothenic acid and betaine; propellants, for example propane, butane, dimethyl ether, N₂O and carbon dioxide.

The afore-said constituents are employed in amounts commonly used for such purposes, for example water in an amount from 0.1 to 95 wt. %, the wetting agents and emulsifiers at a total concentration from 0.2 to 30 wt. %, the alcohols in a total amount from 0.1 to 50 wt. %, the opacifiers, perfume oils, preservatives and dyes in an amount from 0.01 to 5 wt. % each, the buffering agents in a total amount from 0.1 to 10 wt. %, the dissolution promoters, stabilizers, hair-conditioning and hair-care constituents in an amount of 0.1 to 5 wt. % each, the thickeners and dissolution promoters possibly being contained in the said agent in a total amount from 0.5 to 20 wt. %.

The pH of the agent is preferably 2.0 to 10.0, more preferably 3.0 to 9.0 and particularly 3 to 6. If necessary, the desired pH can be attained by addition of an acid, for example an α-hydroxycarboxylic acid such as lactic acid, tartaric acid, citric acid or malic acid, phosphoric acid, acetic acid, glycolic acid, salicylic acid, glutathione or gluconolactone, or else by addition of alkalinizing agents such as ammonia, alkanolamines, alkylamines, alkali metal hydroxides, ammonium hydroxides, alkali metal carbonates, ammonium carbonates or alkali metal phosphates.

For the hair treatment, the agent may be left in the hair or rinsed off after use. In the latter case, the time of exposure to the agent is 10 seconds to 60 minutes and particularly 5 to 20 minutes, depending on the temperature (about 20 to 60 degrees centigrade and preferably 30 to 50 degrees centigrade), it being possible to accelerate the repair activity (sealing, hardening, restructuring) by supplying heat. The use of heat is therefore pre-ferred. At the end of the exposure period, the hair can be rinsed with water and optionally washed with a shampoo.

The forms in which the agent is provided and which are suitable for use according to the invention are preferably shampoos, rinses, conditioners, foams, firming compositions, hair gels, hair sprays, hair colorants, hair tinting agents, permanent wave agents, permanent wave pretreatment agents, fixatives, hair-smoothing agents or brilliantines.

The use according to the invention relates to the application before, during and after a cosmetic hair treatment.

Preferably, the use according to the invention comprises the post-treatment following chemical and/or physical hair treatments, particularly hair dyeing, hair tinting or hair bleaching, or following permanent hair deformation, for the purpose of repairing hair damage and sealing the hair.

We have found that the use according to the invention of an agent containing at least one silane of formula (I) results in definite structural improvement and sealing of previously damaged keratinic fibers that manifests itself in increased tear strength.

Hence, the object of the present invention is, in particular, the use of the silanes described herein for the improvement of the condition of hair following a hair coloring treatment.

A preferred method according to the invention for coloring and at the same time improving the condition of human hair is characterized in that

(a) for 5 to 40 minutes, the hair is brought in contact with a direct hair colorant or an oxidative hair colorant,

(b) the hair colorant is optionally rinsed off with water, and the hair is optionally dried,

(c) the hair is then brought in contact for 1 to 40 min with a silane-containing agent in accordance with one of use claims 1 to 18, and

(d) the hair is optionally rinsed with water and optionally dried.

The improvement in the condition of the hair can also take place if the hair colorant (oxidative or temporary) contains the silane of formula (I). This method of the invention for coloring and at the same time improving the condition of human hair is characterized in that the hair is brought in contact with a direct hair colorant (hair tinting agent) or an oxidative hair colorant for 1 to 40 min, said colorant containing from 0.01 to 10 wt. % of a silane according to formula (I), and the hair is finally washed or rinsed and then dried.

The agent of the invention for oxidative coloring of human hair, containing at least one oxidative hair dye and characterized in that it contains from 0.01 to 10 wt. % of at least one silane of formula (I) or a salt thereof, is also new and therefore an object of the invention.

The hair can either be colored or tinted with “direct” hair dyes only on the surface, or it can be permanently dyed also in the interior of the hair with oxidative dyes. The use according to the invention relates to both hair-coloring methods.

In the case of oxidative coloring, the hair is treated with a hair colorant obtained by mixing a special dye carrier composition based on a developer-coupler combination with a special oxidant preparation.

The first component is referred to as the dye carrier composition and contains the dyes. It can be a liquid, a gel or an emulsion. The second component is an aqueous, liquid, cream-like or powdered oxidant preparation.

The coloring takes place by reaction of certain developers with certain couplers in the presence of an appropriate oxidant, for example hydrogen peroxide.

In dyeing practice, the dye carrier composition (component A) is mixed before use with an aqueous hydrogen peroxide solution, emulsion or cream (component B), for example in a mixing ratio of dye carrier composition to hydrogen peroxide composition of 1:1 to 1;2, for example in an application bottle from which, after the mixing, the ready-to-use oxidative colorant is applied to the hair to be colored.

The dye carrier composition of component (A) contains as oxidative hair dyes at least one coupler and at least one developer and optionally, in addition, also self-coupling dye pre-cursors and optionally, for shading, in addition, also direct hair dyes.

The developers and couplers are used in the hair colorants either in the form of the free base or in the form of a physiologically harmless salt thereof with an inorganic or organic acid, for example as chloride, sulfate, phosphate, acetate, propionate, lactate or citrate.

The developers and couplers are preferably used in approximately equimolar amounts. It is not disadvantageous, however, if the couplers are used in an excess or deficiency relative to the developer, the developer component possibly consisting of a mixture of known developers and the coupler component possibly consisting of a mixture of known couplers.

Suitable developers are, for example:

1,4-diaminobenzene (p-phenylenediamine), 1,4-diamino-2-methylbenzene (p-toluylenediamine), 2,5-diaminoanisole, 2,5-diaminobenzyl alcohol, 1,4-diamino-2,6-dimethylbenzene, 1,4-diamino-2,5-dimethylbenzene, 1,4-diamino-2,3-dimethylbenzene, 2-chloro-1,4-diaminobenzene, 4-phenylaminoaniline, 4-dimethylaminoaniline, 4-diethylaminoaniline, 4-[di(2-hydroxyethyl)amino]aniline, 4-[(2-methoxyethyl)amino]aniline, 4-[(3-hydroxypropyl)-amino]aniline, 1,4-diamino-2-(2-hydroxyethyl)benzene, 1,4-diamino-2-(1-methylethyl)benzene, 1,3-bis[(4-aminophenyl)-(2-hydroxyethyl)amino]-2-propanol, 1,8-bis(2,5-diaminophenoxy)-3,6-dioxaoctane, 4-aminophenol, 4-amino-3-methylphenol, 4-methyl-aminophenol, 4-amino-2-(aminomethyl)phenol, 4-amino-2-[(2-hydroxyethyl)amino]methylphenol, 4-amino-2-(methoxymethyl)phenol, 4-amino-2-(2-hydroxyethyl)phenol, 5-aminosalicylic acid, 2,5-diaminopyridine, 2,4,5,6-tetraminopyrimidine, 2,5,6-triamino-4-(1H)-pyrimidone, 4,5-diamino-1-(2-hydroxyethyl)-1H-pyrazole, 4,5-diamino-1-(1-methylethyl)-1H-pyrazole, 4,5-diamino-1-[(4-methylphenyl)methyl]-H-pyrazole, 1-[(4-chlorophenyl)methyl]4,5-diamino-1H-pyrazole, 4,5-diamino-1 methyl-1H-pyrazole, 2-aminophenol, 2-aminomethylphenol and 2-amino-5-methylphenol, alone or in admixture with one another.

Suitable couplers are, for example:

N-3-dimethylaminophenyl)urea, 2,6-diaminopyridine, 2-amino-4[(2-hydroxyethyl)amino]-anisole, 2,4-diamino-1-fluoro-5-methylbenzene, 2,4-diamino-1-methoxy-5-methylbenzene, 2,4-diamino-1-ethoxy-5-methylbenzene, 2,4-diamino-1-(hydroxyethoxy)-5-methylbenzene, 2,4-[(2-hydroxyethyl)amino]-1,5-dimethoxybenzene, 2,3-diamino-6-methoxy-pyridine, 3-amino-6-methoxy-2-(methylamino)pyridine, 2,6-diamino-3,5-dimethoxypyridine, 3,5-diamino-2,6-dimethoxypyridine, 1,3-diaminobenzene, 2,4-diamino-1-(2-hydroxyethoxy)benzene, 1-(2-aminoethoxy)-2,4-diaminobenzene, 2-amino-1-(2-hydroxyethoxy)-4-methylaminobenzene, 2,4-diaminophenoxyacetic acid, 3-[di(2-hydroxyethyl)amino]aniline, 4-amino-2-di[(2-hydroxyethyl)amino]-1-ethoxybenzene, 5-methyl-2-(1-methylethyl)phenol, 3-[(2-hydroxy-ethyl)amino]-aniline, 3-[(2-aminoethyl]o-amino]aniline [sic], 1,3-di-(2,4-diaminophenoxy)-propane, di(2,4-diaminophenoxy)methane, 1,3-diamino-2,4-dimethoxybenzene, 2,6-bis(2-hydroxyethyl)aminotoluene, 4-hydroxyindole, 3-dimethylaminophenol, 3-diethylaminophenol, 5-amino-2-methylphenol, 5-amino-4-fluoro-2-methylphenol, 5-amino-4-methoxy-2-methylphenol, 5-amino-4-ethoxy-2-methylphenol, 3-amino-2,4-dichlorophenol, 5-amino-2,4-dichlorophenol, 3-amino-2-methylphenol, 3-amino-2-chloro-6-methylphenol, 3-amino-phenol, 2[(3-hydroxyphenyl)amino]acetamide, 5-[(2-hydroxyethyl)amino]-2-methylphenol, 3-[(2-hydroxyethyl)amino]phenol, 3-[(2-methoxyethyl)amino]phenol, 5-amino-2-ethylphenol, 2-(4-amino-2-hydroxyphenoxy)ethanol, 5-[(3-hydroxypropyl)amino]-2-methylphenol, 3-[(2,3-dihydroxypropyl)amino]-2-methylphenol, 3-[(2-hydroxyethyl)amino]-2-methylphenol, 2-amino-3-hydroxypyridine, 5-amino-4-chloro-2-methylphenol, 1-naphthol, 1,5-dihydroxynaphthalene, 1,7-dihydroxynaphthalene, 2,3-dihydroxynaphthalene, 2,7-dihydroxynaphthalene, 2-methyl-1-naphthol acetate, 1,3-dihydroxybenzene, 1-chloro-2,4-dihydroxybenzene, 2-chloro-1,3-dihydroxybenzene, 1,2-dichloro-3,5-dihydroxy-4-methylbenzene, 1,5-dichloro-2,4-dihydroxybenzene, 1,3-dihydroxy-2-methylbenzene, 3 4-methylenedioxyphenol. 4-(β-hydroxyethylamino)-1,2-methylenedioxybenzene, 3,4-methylenedioxyaniline, 5-[(2-hydroxyethyl)-amino]-1,3-benzodioxol, 5-hydroxy-1,3-benzodioxol, 5-amino-1,3-benzodioxol, 4-methoxy-1-naphthol, 2-methyl-1,3-dihydroxybenzene, 6-bromo-1-hydroxy-3,4-methylenedioxybenzene, 3,4-diaminobenzoic acid, 3,4-dihydro-6-hydroxy-1,4-(2H)-benzoxazine, 6-amino-3,4-dihydro-1,4(2H)-benzoxazine, 3-methyl-1-phenyl-5-pyrazolone, 5,6-dihydroxyindole, 5,6-dihydroxyindoline, 5-hydroxyindole, 6-hydroxyindole, 7-hydroxyindole, 2,3-indolinedione, 2,4-dihydroxyphenyl ether such as 2,4-dihydroxyanisole and 2,4-dihydroxyphenoxyethanol.

The total amount of the developer-coupler combination contained in component (A) is from about 0.01 to 12.0 weight percent and particularly from about 0.2 to 4.0 weight percent.

To achieve certain color shades, component (A) can also contain common direct dyes, for example triphenylmethane dyes such as Diamond Fuchsin (C.I. 42 510) and Leather Ruby HF (C.I. 42 520), aromatic nitro dyes such as 2-amino-4,6-dinitrophenol, 2-nitro-4-N-hydroxyethylamino)aniline, 2-N-β-dihydroxypropylamino-54N-methyl,N-hydroxyethyl)-aminonitrobenzene and 2-amino-4-nitrophenol, azo dyes such as Acid Brown 4 (C.I. 14 805) and Acid Blue 135 (C.I. 13 385), anthraquinone dyes such as Disperse Violet 4 (C.I. 61 105), Disperse Blue 1 (C.I. 64 500), Disperse Red 15 (C.I. 60 710), Disperse Violet 1 (C.I. 61 100), 1,4,5,8-tetraminoanthraquinone and 1,4-diaminoanthraquinone.

Moreover, component (A) can also contain self-coupling dye precursors, for example 2-amino-5-methylphenol, 2-amino-6-methylphenol, 2-amino-5-ethoxyphenol or 2-propylamino-5-aminopyridine.

The total amount of direct dyes and self-coupling dye precursors in component (A) is about 0.01 to 7.0 weight percent and preferably about 0.2 to 4.0 weight percent.

Component (A) also contains a combination of nonionic and anionic surface-active compounds (known as surfactants or emulsifiers), preferably in a total amount from about 0.1 to 25 weight percent and particularly from about 1.0 to 20 weight percent. Suitable nonionic surface-active compounds are, in particular, ethoxylated C₁₀-C₂₀ fatty alcohols, (C₁₂-C₁₈) partial esters of ethoxylated sorbitan fatty acids, (C₁₂-C₁₈) partial esters of ethoxylated glycerol fatty acids or mixtures of these compounds, having a degree of ethoxylation preferably from 1 to 30 and particularly from 2 to 6, or C₁₂-C₁₈-alkylglucosides. Suitable anionic surface-active compounds are, in particular, alkylsulfates, alkyl ether sulfates with a C₁₀-C₂₀ alkyl group or mixture thereof, having a degree of ethoxylation preferably from 1 to 8 and particularly from 1 to 6. Suitable amino acids are the neutral as well as the acidic or basic amino acids. Examples of suitable amino acids are glycine, alanine, valine, leucine, glutamic acid and arginine. The total amount of amino acids in the agent of the invention is preferably from 0.01 to 5 weight percent.

Component (A) preferably also contains monohydric or dihydric alcohols, for example low-molecular-weight C₁-C₄ alcohols or C₂-C₆ glycols, for example ethanol, propanol, isopropanol, ethylene glycol, 1,2-propylene glycol or butylene glycol, or a mixture thereof, said alcohols preferably being used in an amount from about 1 to 40 weight percent and particularly from about 5 to 30 weight percent.

Optionally, it is possible to add to component (A) special hair-care substances and/or active materials, for example protein hydrolyzates, such as keratin hydrolyzates, elastin hydrolyzates, collagen hydrolyzates, silk protein hydrolyzates, milk protein hydrolyzates, soy protein hydrolyzates or wheat protein hydrolyzates, panthenol; allantoin; pyrrolidonecarboxylic acids or salts thereof; plant extracts or vitamins; or cationic polymers alone or in combination with one another, said compounds preferably being contained in component (A) at a total concentration of about 0.01 to 5 weight percent and particularly about 0.01 to 1 weight percent.

Moreover, component (A) can contain antioxidants, for example ascorbic acid, thioglycolic acid or sodium sulfite, or complexing agents for heavy metals, for example ethylenediamine tetraacetate or nitrilotriacetic acid, in an amount of up to about 0.5 weight percent. Perfume oils can be contained in component (A) in an amount of up to about 1 weight percent.

Component (B) contains as the oxidant for developing the hair color hydrogen peroxide or a product of addition thereof to urea, melanin [sic] or sodium borate, the oxidant preferably being used in an amount from about 1 to 18 weight percent and particularly from about 4 to 14 weight percent. Particularly preferred is the use of hydrogen peroxide in an amount from 1 to 18 weight percent.

If the oxidant-containing component (B) is in the form of an emulsion, it can advantageously contain a fatty alcohol of either natural or synthetic origin, C₁₄-C₂₀ fatty alcohols, particularly cetyl alcohol and stearyl alcohol or a mixture thereof, being preferred. The amount of fatty alcohol used in component (B) is about 2 to 15 weight percent and particularly about 5 to 12 weight percent. Component (B) preferably contains about 0.1 to 5 weight percent and particularly about 0.3 to 3 weight percent of a polypropylene glycol ether, polyethylene glycol ether or polyethylene glycol/polypropylene glycol ether. Suitable polypropylene glycol ethers, polyethylene glycol ethers or polyethylene glycol/polypropylene glycol ethers are, for example, PPG-16 Butyl Ether, PPG-1-PEG-9 Lauryl Glycol Ether, PPG-38 Buteth-37 or PPG-1 PEG-9 Lauryl Ether. Advantageously, component (B) also contains about 0.5 to 10 weight percent, but preferably about 1 to 7 weight percent, of Acrylates/Steareth-20 Itaconate Copolymer or Acrylates/Ceteth-20 Itaconate.

In all cases, the afore-indicated percentages are based on the total amount of component (A) or on the total amount of component (B).

The hair colorants for temporary dyeing or tinting of hair with direct hair dyes is equal in composition to component (A), but contains as hair dyes no oxidative dyes or self-coupling dye precursors, but only one or more of the aforesaid or other direct dyes in the amounts indicated.

The method of the invention for improving the condition of hair after hair dyeing consists of mixing the dye carrier composition [component (A)] just before use with the hydrogen peroxide composition [(component (B)] in a weight ratio of 1:1 to 1:4 and preferably in a weight ratio of 1:1 to 1:3, or of using the temporary hair colorant directly by applying an amount of the ready-to-use hair colorant to previously washed hair in an amount sufficient for hair dyeing, in general from 60 to 160 g, depending on the fullness of the hair. After an exposure time of about 5 to 40 minutes, preferably about 20 to 30 minutes, at 15 to 50 degrees centigrade, the hair is rinsed with water, optionally washed with a shampoo and preferably dried. The hair is then treated with the afore-described silane-containing agent.

An advantageous embodiment of the invention concerns the use of silanes of formula (I) or the salts thereof, preferably in an amount from 0.01 to 10 wt. % and particularly in an amount from 0.1 to 5 wt. %, in a permanent wave pretreatment agent. Before applying the permanent wave agent (reducing agent and oxidative fixation), the permanent wave pretreatment agent is brought in contact with the hair, particularly with the hair tips. The silane preferably seals the highly damaged sites of the hair—particularly the hair tips—thus protecting them from the action of the subsequently used reducing permanent wave agent.

Unless otherwise indicated, all percentages given in the present Description are weight percentages based on the total weight of the particular composition involved.

Testing

Determination of the Amount of Hair Dye Removed by Hair Washing

Bleached, permanently waved human hair strands were first dyed with the commercial oxidative colorant Koleston Perfect Nuancen 4/6, 6/45 or 8/46 (manufacturer: Wella AG, Darmstadt) in a manner in and of itself known. The color intensity (E^{o}) was measured on dried hair with the commercial Minolta ChromaMeter II Reflectance color-measuring instrument. Then, a 1%, 3% or 5% aqueous solution of 1H,1H,2H,2H-perfluorooctyltriethoxysilane (PFOS) was applied to the hair either without previous acidification of the hair with citric acid solution (Tests. 2 to 4) or with acidification of the hair with 2% citric acid solution (Tests 5 to 7). After an exposure time of 15 minutes, the hair was rinsed with water and then dried. The hair was then washed for 5 min with a commercial shampoo, rinsed with water and again dried. The color intensity E was then measured once again. The following Table 1 shows the calculated color intensity differences (ΔE) between the initial color intensity (E^{o}) and the color intensity after the hair washing (E) as a function of the silane solution concentration used. For comparison, Test 1 shows the result obtained with a strand that had not been treated with a silane solution after the dyeing (standard).

Table 1 shows that, compared with the standard that had not been treated with a silane solution (Test 1), the color removal was drastically reduced when after an oxidative hair dyeing the hair was sealed with a silane solution (Tests. 2 to 7). This resulted in excellent color stability.

TABLE 1
Determination of the Resistance of Hair
Coloration to Removal by Washing

| | | Color Difference | | |
|---|---|---|---|---|
| | | ΔE | ΔE | ΔE |
| | | Koleston | | |
| Test | Silane | Perfect Nuance | | |
| No. | Concentration | 4/6 | 6/45 | 8/46 |
| 1 | zero | 10.69 | 16.54 | 6.33 |
| 2 | 1% | 7.79 | 7.59 | 3.74 |
| 3 | 3% | 7.35 | 7.14 | 2.67 |
| 4 | 5% | 4.12 | 5.56 | 3.49 |
| 5 | 1% | 3.34 | 1.87 | 0.81 |
| 6 | 3% | 2.65 | 2.36 | 1.31 |
| 7 | 5% | 1.64 | 2.43 | 0.65 |

Determination of the Repair Activity

The organosilane, for example the preferred fluorosilane, was dissolved in isopropanol and prehydrolyzed with water in the following Tests 1 and 4 (Table 2). In each case, the hair was then treated for 1 minute with the organosilane-containing preparation. The hair coated in this manner was then dried at room temperature.

The breaking strength of the hair, which is an indicator for the structural integrity of the hair cortex and, hence, a measure of the degree of damage, was for these purposes determined by running the conventional tensile-elongation measurements. Twenty individual hairs were selected from each hair strand, and the individual hair diameters were determined with a computer-controlled laser micrometer. Then, with a tensile-elongation tester (MTT 160/600 Series Miniature Tensile Tester, Serial No. 600.95.05.001, manufacturer: DIA-STRON Ltd., England), the force needed to cause the individual hairs to break was measured. From these individual breaking strength values which differed from one another because of the different hair diameters, the bundle tensile strength (BTS) was determined by calculating from the individual values and taking into account the particular hair diameter the breaking strength for a hair diameter of 0.08 mm (mean diameter). Finally, by taking into account the hair density, the conversion into the unit of bundle tensile strength (cN/tex) was made. The higher the numerical value of the bundle tensile strength the lower is the hair damage.

We have found that the addition of an organosilane brings about marked hair strengthening or repair action.

Determination of the Improvement in Combability

Hair combability is also an important parameter for describing the condition of hair. Different external influences, such as certain cosmetic treatments (bleaching, dyeing, permanent waves), weathering, frequent combing and brushing degrade the combability of the hair as a result of damage to the cuticula.

The principle of most methods for determining combability consists of measuring the force (combing force) required to pass a comb through a strand of hair under exactly defined boundary conditions.

In our own study, we used for this purpose an automatized apparatus in which a mechanical gripping arm brought the strands to be investigated from storage and suspended them on the hook of a force-measuring cell. The strands were then repeatedly and automatically combed at constant rate, and for each combing step the combing force N (newton) was recorded as a function of the combing path (strand length). The indicated combing force values were finally obtained by averaging the combing forces over the combing path. The lower the combing force, the better is the combability of the hair.

The measurements carried out on hair that had been treated with silane-containing and silane-free shampoos gave the following results.

Damaged [blonded] hair was treated with a shampoo as in Example 2, but without organosilane (the organosilane was replaced with the same quantity of water). A 1 wt. % content of organosilane reduced the combing force by about 8%. The addition of an organosilane (PFOS) thus produced a definitely detectable improvement in wet combability of the hair.

The organofluorosilanes 1H, 1H,2H,2H-perfluorooctyltriethoxysilane (PFOS) and 1H,1H,2H,2H-perfluorodecyltriethoxysilane (PFDS) were dissolved in isopropanol and for samples 1 and 4 prehydrolyzed with water for 1 hour. To each silane solution was added one-half of the stoichiometric amount of water. The tests were made with hair bleached for different lengths of time. The bleaching time was 5 to 30 minutes. The hair strands were completely immersed into the silane solution to exclude any influence of the immersion depth on the results of the subsequently performed measurements of combing force and luster. The coated hair was then dried at room temperature. For strands 1 to 12, the water absorption capacity was then determined. These strands were additionally examined with the scanning electron microscope. On strands 13 to 20, combing force and luster measurements were carried out.

The applied coating treatments are presented in the following Tables 2 and 3.

TABLE 2
Coating of Hair Strands with Organofluorosilanes

| Hair Sample | Bleaching | | Silane Content | |
|---|---|---|---|---|
| No. | Time, min | Silane | of Isopropanol | Condition |
| 1 | 30 | PFOS | 10% | silane prehydrolyzed |
| 2 | 30 | PFOS | 1% | hair moistened |
| 3 | 30 | PFOS | 1% | hair dry |
| 4 | 30 | PFOS | 1% | silane prehydrolyzed |
| 5 | 30 | PFDS | 0.5% | hair dry |
| 6 | 30 | PFDS | 1% | hair dry |
| 7 | 30 | PFDS | 2% | hair dry |
| 8 | 5 | PFDS | 1% | hair dry |
| 9 | 10 | PFDS | 1% | hair dry |
| 10 | 15 | PFDS | 1% | hair dry |
| 11 | 20 | PFDS | 1% | hair dry |
| 12 | 25 | PFDS | 1% | hair dry |

TABLE 3
Coating of Bleached and Unbleached Hair Strands

| Hair Sample | | | Silane Content |
|---|---|---|---|
| No. | Condition | Silane | of Isopropanol |
| 13 | unbleached | PFDS | 0.1% |
| 14 | bleached | PFDS | 0.1% |
| 15 | unbleached | PFDS | 1% |
| 16 | bleached | PFDS | 1% |
| 17 | unbleached | PFOS | 0.1% |
| 18 | bleached | PFOS | 0.1% |
| 19 | unbleached | PFOS | 1% |
| 20 | bleached | PFOS | 1% |

Characterization

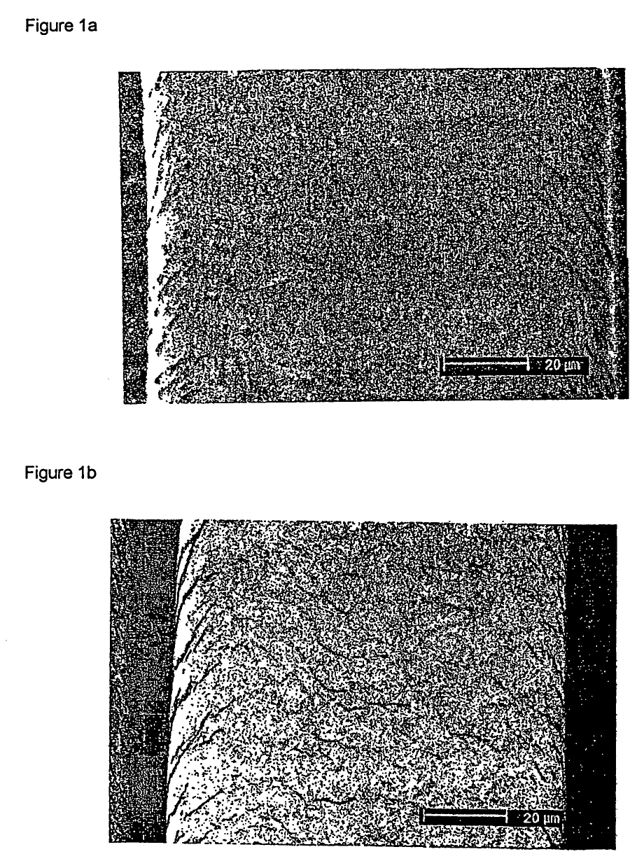

The coated hair was examined under the scanning electron microscope. FIGS. 1a and 1b show a comparison of the photomicrographs of two hair strands treated with prehydrolyzed 1H,1H,2H,2H-perfluorooctyltriethoxysilane. FIG. (1a) shows a strand for which 10% of 1H,1H,2H,2H-perfluorooctyltriethoxysilane was added to the solution (hair sample 1). For the strand shown in FIG. (1b), only 1% of the perfluorooctyltriethoxysilane was used (hair sample 4). Both photomicrographs show that the silane is very well adsorbed by the hair and on the hair surface forms a closed film that becomes appreciably thicker as the concentration is increased.

Determination of Water Absorption Capacity

Before the measurement, the coated hair was first dried 1 hour at 50° C. in an oven. It was then weighed to determine its 100% weight. The hair strands were then immersed in city water to a distance of 1 cm from their upper end. They were then made to swing for 10 seconds, allowed to drain, and their tips were then dabbed on kitchen paper. The hair was then again weighed. A second or third weighing was done after 20 and 45 minutes, respectively. At the end, the hair was dried with kitchen paper and once again weighed.

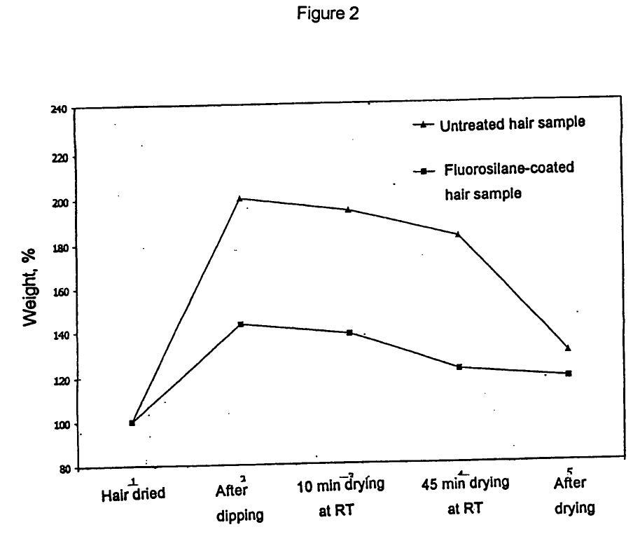

FIG. 2 shows a plot of the water absorption capacity of an untreated hair strand (top curve) in comparison with a strand coated with the organosilane (bottom curve).

In this case, the reduced water absorption of the coated hair is quite evident. Treatment with the organosilane resulted in the absorption of only 40% of the amount of water absorbed by the untreated hair. As a result, the drying time was also reduced. During drying with a hair drier, hair treated in this manner is exposed to less stress, because the duration of drying and the drying temperature can be reduced.

The dependence of this hydrophobing effect on the concentration of the silane used and on the duration of the hair bleaching was then examined. As indicated by scanning electron microscope studies, the film thickness of the silane on the hair can be increased by increasing the silane concentration (cf. FIGS. 1a and 1b). Measurement of the water absorption capacity has shown that a higher silane content causes the water absorption to drop as result of a more complete coverage of the hair surface. The shape of the curve in

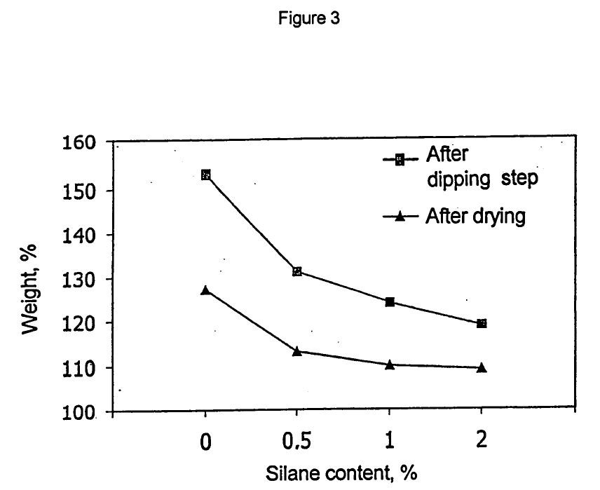

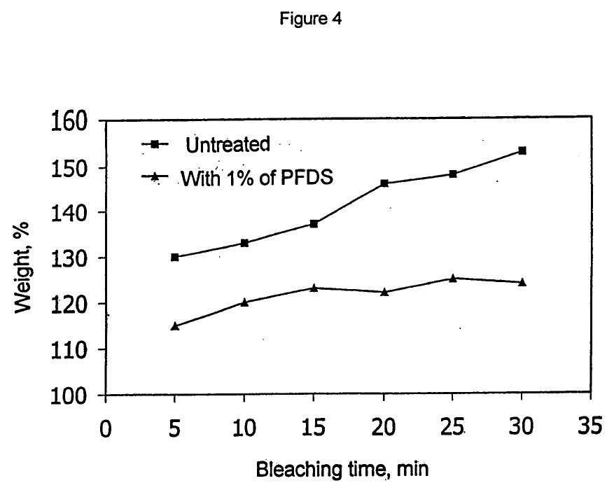

FIG. 3 suggests saturation taking place when the hair surface is completely covered with the silane.

The treatment of hair with an organosilane as the hydrophobing component brings about a reduced water absorption by the hair. The drying time is thereby shortened, and the swelling of the hair, for example in humid weather, is reduced. The combability and the luster of the hair are improved by the organosilane treatment. The silanes are adsorbed by the hair very uniformly and form a closed film on the hair surface. As a result of this property, they are very well suited for sealing previously dyed hair and thus for preventing the dye from being washed out or from “bleeding out”. When used after the hair has been dyed, the silanes improve not only the condition of the hair but also ensure a longer-lasting hair coloration. The resistance of the dyed hair to perspiration, light, permanent waves and washing, in particular, is clearly improved.

The following examples will explain the subject matter in greater detail.

### EXAMPLES

### Example 1

### Hair Spray

| | | |
|---|---|---|
| Vinyl acetate/crotonic acid copolymer | 2.00 | wt. % |
| 2-Amino-2-methyl-1-propanol | 0.16 | wt. % |
| Isopropanol | 37.84 | wt. % |
| 1H,1H,2H,2H-Perfluorooctyltriethoxysilane (PFOS) | 1.50 | wt. % |
| Perfume oil | 0.10 | wt. % |
| Propane/butane | to 100.00 | wt. % |

### Example 2

### Hair Shampoo

| | | |
|---|---|---|
| Sodium lauryl ether sulfate (25% aqueous solution) | 40.0 | wt. % |
| NaCl | 4.0 | wt. % |
| 1H,1H,2H,2H-Perfluorooctyltriethoxysilane (PFOS) | 1.0 | wt. % |
| Water | to 100.0 | wt. % |

### Example 3

### Hair Shampoo

| | | |
|---|---|---|
| Sodium lauryl ether sulfate (25% aqueous solution) | 35.0 | wt. % |
| NaCl | 3.0 | wt. % |
| Triethanolamine | 4.0 | wt. % |
| 1,2-Dibromo-2,4-dicyanobutamine-2-phenoxyethanol | 0.1 | wt. % |
| Perfume oil | 0.1 | wt. % |
| 1H,1H,2H,2H-Perfluorodecyltriethoxysilane (PFDS) | 2.0 | wt. % |
| Water | to 100.0 | wt. % |

### Example 4

### Hair Treatment

| | | |
|---|---|---|
| Glycerol monostearate | 6.0 | wt. % |
| Lanoline alkoxylate | 2.0 | wt. % |
| Cetyl alcohol | 2.0 | wt. % |
| Mixture of lanolin alcohol and paraffin oil | 1.0 | wt. % |
| Tris(oligooxyethyl)alkylammonium phosphate | 1.5 | wt. % |
| Hydroxyethylcellulose | 20.0 | wt. % |
| Citric acid | 0.1 | wt. % |
| Sorbic acid | 0.1 | wt. % |
| Perfume oil | 0.1 | wt. % |
| 1H,1H,2H,2H-Perfluorooctyltriethoxysilane (PFOS) | 2.0 | wt. % |
| Water | to 100.0 | wt. % |

### Example 5

### Foam Conditioner

| | | |
|---|---|---|
| PVP/vinylimidazolium methochloride copolymer | 5.00 | wt. % |
| PVP/PVA copolymer | 1.00 | wt. % |
| Polyoxyethylene-12-cetylstearyl alcohol | 0.15 | wt. % |
| Perfume oil | 0.10 | wt. % |
| 1H,1H,2H,2H-Perfluorodecyltriethoxysilane (PFDS) | 1.00 | wt. % |
| Propane/butane | 10.00 | wt. % |
| Water | to 100.00 | wt. % |

### Example 6

### Brilliantine

| | |
|---|---|
| Candelilla wax | 79.6 wt. % |
| Paraffin oil | 14.8 wt. % |
| Isopropyl myristate | 4.6 wt. % |
| Perfume oil | 0.5 wt. % |
| 1H,1H,2H,2H-Perfluorooctyltriethoxysilane (PFOS) | 0.5 wt. % |
| | 100.0 wt. % |

### Example 7

### Permanent Wave Agent

| | | |
|---|---|---|
| Thioglycolic acid (80% aqueous solution) | 9.5 | wt. % |
| Ammonia (25% aqueous solution) | 1.6 | wt. % |
| Ammonium carbonate | 4.5 | wt. % |
| 1H,1H,2H,2H-Perfluorooctyltriethoxysilane (PFOS) | 2.0 | wt. % |
| Perfume oil | 0.2 | wt. % |
| Water | to 100.0 | wt. % |

### Example 8

### Permanent Wave Fixative

| | | |
|---|---|---|
| Hydrogen peroxide | 4.6 | wt. % |
| Citric acid | 0.2 | wt. % |
| 1H,1H,2H,2H-Perfluorodecyltriethoxysilane (PFDS) | 2.0 | wt. % |
| Perfume oil | 0.1 | wt. % |
| Water | to 100.0 | wt. % |

### Example 9

### Oxidative Colorant in Cream Form

| | | |
|---|---|---|
| Stearyl alcohol | 8.00 | wt. % |
| Paraffin oil | 13.00 | wt. % |
| Wool grease | 6.00 | wt. % |
| Perfume | 0.30 | wt. % |
| p-Toluylenediamine | 0.70 | wt. % |
| Resorcinol | 0.05 | wt. % |
| Aminophenol | 0.06 | wt. % |
| Ethylenediamine tetraacetate (EDTA) | 0.20 | wt. % |
| Ammonia (2% aqueous solution) | 2.00 | wt. % |
| Sodium sulfite | 1.00 | wt. % |
| 1H,1H,2H,2H-Perfluorooctyltriethoxysilane (PFOS) | 1.00 | wt. % |
| Water | to 100.00 | wt. % |

1-23. (canceled)

24. A cosmetic agent for improving a condition of hair, said agent comprising at least one silane of formula I:
RₘSiX₄₋ₘ (I),
wherein m denotes an integer from 0 to 4,
R is a nonhydrolyzable organic group, and
X denotes a hydrolyzable group.

25. The cosmetic agent as defined in claim 24, wherein said integer is from 0 to 3.

26. The cosmetic agent as defined in claim 25, wherein each R, independently of each other, denotes a substituted or unsubstituted alkyl group each with 1 to 16 carbon atoms, a substituted or unsubstituted hydroxyalkyl group each with 1 to 16 carbon atoms and 1 to 6 hydroxyl groups or a substituted or unsubstituted phenyl group; and
each X, independently of each other, denotes a hydroxyl, halogen, acetyl, acetoxy, acyl, acyloxy or alkoxy group with 1 to 6 carbon atoms, a hydroxylated polymer unit, a polyglycol or a polyalkyl ether with 4 to 18 carbon atoms.

27. The cosmetic agent as defined in claim 24, wherein said integer is 1 or 2.

28. The cosmetic agent as defined in claim 24, wherein said at least one silane is at least one of compounds of formula IV, of formula V, of formula VI and of formula VII:
(R1)₃SiR₂N⁺(R3)(R4)(R5)Y⁻ (IV),
(R1)₃SiR2N(R3)(R4) (V),
(R1)₃SiR2R7 (VI),
(R1)₂SiR8R9 (VII),
wherein each R1, independently of each other, is a halogen or R6O, wherein R6 is H; alkyl; aryl, acetyl; acetoxy; acyl; acyloxy; glycol; polyglycol; alkylglycol, alkyl polyglycol or a monoester formed by linking a carboxylic acid with 1 to 24 carbon atoms with a glycol or polyglycol; alkylphenol, substituted with an alkyl with 1 to 24 carbon atoms, an ether thereof or a sorbitan ester thereof;
R7 denotes R6, H, halogen, halogenated or perhalogenated alkyl or aryl, NH₂(CH₂)₂NHR₂, NH₂R₂, C₃H₅O₂R₂, C₄H₆O₂R₂, NaO(CH₃O)P(O)R₂ or ClCH₂C₆H₄R₂;
R8 and R9, independently of each other, denote R7, alkyl with 1 to 24 carbon atoms, isobutyl, phenyl or n-octyl;
R2 denotes R6, benzyl or vinyl;
R3 and R4, independently of each other, each denote R7, hydroxyalkyl, alkoxy or alkyl with 1 to 6 carbon atoms, or R3 and R4 together represent a morpholine group or another cyclic or heterocyclic group;
R5 denotes hydroxyalkyl, R7CH₂C₆H₅, polyglycol, alkyl, alkoxy, perfluoroalkyl, perfluoroalkyl sulfonate or perfluoroalkyl carboxylate; and
Y⁻ is an anion of salts of the compounds of formula IV.

29. The cosmetic agent as defined in claim 24, wherein said at least one silane is selected from the group consisting of 3-(trimethoxysilyl)propyl-dimethyloctadecylammonium chloride, 3-(trimethoxysilyl)propylmethyl-di(decyl)ammonium chloride, 3-chloropropyltrimethylsilane, 3-chloropropyl-trimethoxysilane, octadecyltrimethoxysilane, 1H, 1H, 2H,2H-perfluorooctyl-triethoxysilane and 1H, 1H, 2H, 2H-perfluorodecyltriethoxysilane.

30. The cosmetic agent as defined in claim 24, wherein said at least one silane is selected from the group consisting of NH₂(CH₂)₂NH(CH₂)₃Si(OCH₃)₃, NH₂(CH₂)₃Si(OCH₃)₃, NH₂(CH₂)₃Si(OCH₂CH₃)₃, Cl(CH₂)₃Si(OCH₃)₃, Cl(CH₂)₃Si(OCH₂CH₃)₃, Cl(CH₂)₃SiCl₃, C₃H₅O₂(CH₂)₃Si(OCH₂CH₃)₃, C₃H₅O₂(CH₂)₃Si(OCH₂CH₃)₃, C₄H₅O₂(CH₂)₃Si(OCH₃)₃, C₄H₅O₂(CH₂)₃Si—(OCH₂CH₃)₃, CH₃SiHCl₂, NaO(CH₃O)P(O)(CH₂)₃Si(OH)₃, SiHCl₃, N-2-vinyl-benzylaminoethyl-3-amino-propyltrimethoxysilane.HCl, H₂C═CHSi(OCOCH₃)₃, H₂C═CHSi(OCH₃)₃, H₂C═CHSi(OCH₂CH₃)₃, H₂C═CHSiCl₃, (CH₃)₂SiCl₂, (CH₃)₂Si(OCH₃)₂, (C₆H₅)₂SiCl₂, (C₂H₅)SiCl₃, (C₂H₅)Si(OCH₃)₃, (C₂H₅)Si(OCH₂CH₃)₃, isobutyltrimethoxysilane, n-octyltriethoxysilane, CH₃(C₆H₅)SiCl₂, CH₃SiCl₃, CH₃Si(OCH₃)₃, C₆H₅SiCl₃, C₆H₅Si(OCH₃)₃, C₃H₇SiCl₃, C₃H₇Si(OCH₃)₃, SiCl₄, ClCH₂C₆H₄CH₂CH₂SiCl₃, ClCH₂C₆H₄CH₂CH₂Si(OCH₃)₃, ClCH₂C₆H₄CH₂CH₂Si(OCH₂CH₃)₃, decyltrichlorosilane, dichloromethyl(4-methylphenethyl)silane, diethoxymethylphenylsilane, trimethoxysilane, 3-(dimethoxymethylsilyl)-1-propanethiol, dimethoxymethylvinylsilane, 3-propyl methacrylate, trichlorosilane, methyl-bis-(trimethylsilyloxy)vinylsilane, methyltripropoxysilane and trichlorocyclopentylsilane.

31. The cosmetic agent as defined in claim 24, wherein said at least one silane is a compound of formula VIII:
(Ra)₃SiRb (VIII),
wherein each Ra is, independently of each other, halogen or R10O, wherein R10 is an alkyl group with 1 to 6 carbon atoms, an acetyl group, an acetoxy group, an acyl group, an acyloxy group; a glycol group; a polyglycol group; an alkylglycol group with 1 to 10 carbon atoms or an alkylpolyglycol group with 1 to 10 carbon atoms; and
Rb denotes an alkyl group with 1 to 16 carbon atoms, a halogenated alkyl group with 1 to 16 carbon atoms or a perhalogenated alkyl group of formula CₚZ₂ₚ₊₁ with Z=F, Cl or Br, and p=1 to 16.

32. The cosmetic agent as defined in claim 24, wherein said at least one silane is a compound of formula IX:
(RcO)₃SiRd (IX),
wherein Rc denotes a straight-chain or branched alkyl group with 1 to 5 carbon atoms and Rd denotes a straight-chain or branched fluorinated or perfluorinated alkyl group with 1 to 8 carbon atoms.

33. The cosmetic agent as defined in claim 24, wherein said at least one silane is a compound of formula IX′:
(RcO)₃SiRd (IX′),
wherein Rc denotes a straight-chain or branched alkyl group with 1 to 5 carbon atoms and Rd denotes a straight-chain or branched fluorinated or perfluorinated alkyl group with 8 to 10 carbon atoms.

34. The cosmetic agent as defined in claim 33, wherein said compound of the formula IX′ is 1H,1H,2H,2H-perfluorooctyltriethoxysilane or 1H,1H,2H,2H-perfluorodecyltriethoxysilane.

35. The cosmetic agent as defined in claim 24, further comprising a UV filter linked with said at least one silane by elimination of an atom or group from the UV filter and linking said at least one silane to a site on said UV filter at which said atom or group is eliminated, and wherein said UV filter is para-amino-benzoic acid, aminobenzoic acid, salicylic acid, cinnamic acid, benzoic acid or benzophenone, or an alkyl derivative or anhydride or mixed anhydride thereof.

36. A permanent wave pretreatment agent comprising the cosmetic agent as defined in claim 24.

37. A method of improving a condition of hair, said method comprising bringing the cosmetic agent as defined in claim 24 into contact with the hair.

38. The method as defined in claim 37, wherein said at least one silane, or a salt thereof, is present in the cosmetic agent in an amount of from 0.01 to 10 percent by weight, based on a total amount of the cosmetic agent.

39. The method as defined in claim 37, wherein said at least one silane, or a salt thereof, is present in the cosmetic agent in an amount of from 0.1 to 5 percent by weight, based on a total amount of the cosmetic agent.

40. The method as defined in claim 37, wherein said cosmetic agent is brought into contact with the hair for a period of 10 seconds to 60 minutes at a temperature of between 20° C. and 60° C.

41. The method as defined in claim 37, wherein the improving the condition of the hair consists of at least one of hardening, strengthening, sealing, sealing after hair dyeing or tinting, restructuring, repairing, stabilizing, enhancing luster, enhancing volume, enhancing combability, protecting from environmental influences, protecting from heat, protecting from heat generated by a hair drier, protecting from heat generated by a hot iron during hair smoothing, preventing and reducing hair splitting, shortening the hair-drying time and increasing the tensile strength and elasticity of the hair as well as the permanence of permanent waves.

42. The method as defined in claim 37, wherein said cosmetic agent is brought into contact with the hair after the hair has been subjected to a chemical treatment.

43. The method as defined in claim 42, wherein the chemical treatment comprises dyeing, tinting, bleaching or permanent deformation.

44. The method as defined in claim 37, wherein the hair is damaged, weakened, sensitive, brittle and/or fine.

45. An agent for dyeing and simultaneously improving a condition of human hair, said agent containing a direct hair colorant or an oxidative hair colorant and from 0.01 to 10 percent by weight of at least one silane of formula 1, or a salt thereof:
RₘSiX₄₋ₘ (1),
wherein m denotes an integer from 0 to 4,
R is a nonhydrolyzable organic group, and
X denotes a hydrolyzable group.

46. A method for dyeing and simultaneously improving a condition of human hair, said method comprising bringing the hair in contact with the agent as defined in claim 45 for from 1 to 40 minutes, then washing or rinsing the hair and subsequently drying the hair.

47. A method for dyeing and simultaneously improving a condition of human hair, said method comprising the steps of:
a) bringing the hair into contact with a direct hair colorant or an oxidative hair colorant for 5 to 40 minutes;
b) optionally rinsing the hair colorant out of the hair with water and optionally drying the hair;
c) bringing the hair into contact with a cosmetic agent as defined in claim 24; and
d) optionally rinsing the hair with water and optionally drying the hair.
